SEQUENCE LISTING (9 sequences)

SEQ ID NO: 1 - DNA, 34 bp, Bacteriophage P1
  source 1..34: mol_type = genomic DNA
ataacttcgtatagcatacattatacgaagttat

SEQ ID NO: 2 - DNA, 870 bp, synthetic construct
  misc_feature 1..870: note = hTSHRa
  source 1..870: mol_type = other DNA
atgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaacggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagaaacttccactttccttgagtttccttcacctcacacgggctgacctttcttacccaagccactgctgtgcttttaagaatcagtaa

SEQ ID NO: 3 - DNA, 15501 bp, synthetic construct
  misc_feature 1..15501: note = Rosa26_hTSHRa_vector
  source 1..15501: mol_type = other DNA
ccccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggacgcttccggccgacgtctcgtcgctgattggcttttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctgacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttgccccctcttcccctcgtgatctgcaactccagtctttctagcatctgtagggcgcagtagtccagggtttccttgatgatgtcatacttatcctgtcccttttttttccacagctcgcggttgaggacaaactcttcgcggtctttccagtggttaattaagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagttaattaaataacttcgtatagcatacattatacgaagttatggatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgcaaaaccacactgctcgactagtgattaaagtcgactcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctcatgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttataacttcgtatagcatacattatacgaagttatcggcgcgccgccaccatgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaacggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagaaacttccactttccttgagtttccttcacctcacacgggctgacctttcttacccaagccactgctgtgcttttaagaatcagtaacccggggggatccactagttctagagcggccgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccgcgccgatatcgaattcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataggaaaatggaggataggagtcatctggggtttatgcagcaaaactacaggtatattgcttgtatccgcctcggagatttccatgaggagataaagacatgtcacccgagtttatactctcctgcttagatcctactacagtatgaaatacagtgtgcgaggtagactatgtaagcagatttaatcattttaaagagcccagtacttcatatccatttctcccgctccttctgcagccttatcaaaaggtatttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccattataactgagctataccattaatggtttcaggtggatgtctcctcccccaatatacctgatgtatctacatattgccaggctgatattttaagacataaaggtatatttcattattgagccacatggtattgattactgctactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataatcctccttcttcttctttttttttttttaaagagaatctgtctccagtagaattactgaagtaatgaaatactttgtgtttgttccaatatggagccaataatcaaatactcttagcactggaaatgtaccaaggaactattttatttaagtgactgtggacagaggagccataactgcagacttgtgggatacagaagaccaatgcagacttaatgtcttttctcttacactaagcaataaagaaataaaaattgaacttctagtatcctatttgttaaactgctagctttactaacttttgtgcttcatctatacaaagctgaaagctaagtctgcagccattactaaacatgaaagcaagtaatgataattttggatttcaaaaatgtagggccagagtttagccagccagtggtggtgcttgcctttatgccttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttcttaaagcctccttccctgccatgtggtctcttgtttgctactaacttcccatggcttaaatggcatggctttttgccttctaagggcagctgctgagtttgcagcctgatttccagggtggggttgggaaatctttcaaacactaaaattgtcctttaatttttttttaaaaaatgggttatataataaacctcataaaatagttatgaggagtgaggtggactaatattaatgagtccctcccctataaaagagctattaaggctttttgtcttatactaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgactttaatgttatttgctactgtgaatataaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaatgtgtttaagtgatgaaaacttgaattattatcaccgcaacctactttttaaaaaaaaaagccaggcctgttagagcatgctaagggatccctaggacttgctgagcacacaagagtagtacttggcaggctcctggtgagagcatatttcaaaaaacaaggcagacaaccaagaaactacagtaaggttacctgtctttaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggttatactcagaagcacttctttgggtttccctgcactatcctgatcatgtgctaggcctccttaggctgattgttgttcaaataacttaagtttcctgtcaggtgatgtcatatgatttcatatatcaaggcaaaacatgttatatatgttaaacatttgacttaatgtgaaagttaggtctttgtgggttttgattttaatttcaaaacctgagctaaataagtcattttacatgtcttacatttggtgaattgtatattgtggtttgcaggcaagactctctgacctagtaaccctcctatagagcactttgctgggtcacaagtctaggagtcaagcatttcaccttgaagttgagacgttttgttagtgtatactagttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgtatataactataaccaatactaactccactgggtggatttttaattcagtcagtagtcttaagtggtctttattggcccttattaaaatctactgttcactctaacagaggctgttggactagtggactaagcaacttcctacggatatactagcagataagggtcagggatagaaactagtctagcgttttgtatacctaccagcttatactaccttgttctgatagaaatatttaggacatctagcttatcgatccgtcgacggtatcgataagcttgatatcgaattctaccgggtaggggaggcgcttttccaaggcagtctgagcatgcgcttagcagccccgctggcacttggcgctacacaagtggccttggcctcgcacacattccacatccaccggtaggcgccaaccggctccgttctttggtggccccttcgcgccaccttctctcctcccctagtcaggaagttcccccccgccccgcagctcgcgtcgtaggacgtgacaaatggaagtagcacgtctcactagtctcgtcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgatgatgttgttattcttctaatcttttgtatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgacctcgagggggggcccgcggccgccctgcagggcgatcgctacgtacagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 4 - DNA, 21 bp, synthetic construct
  misc_feature 1..21: note = Rosa26 F primer
  source 1..21: mol_type = other DNA
aaagtcgctctgagttgttat

SEQ ID NO: 5 - DNA, 21 bp, synthetic construct
  misc_feature 1..21: note = Rosa26 R primer
  source 1..21: mol_type = other DNA
ggagcgggagaaatggatatg

SEQ ID NO: 6 - DNA, 20 bp, synthetic construct
  misc_feature 1..20: note = hTSH-A F primer
  source 1..20: mol_type = other DNA
gcaacgtgctggttattgtg

SEQ ID NO: 7 - DNA, 21 bp, synthetic construct
  misc_feature 1..21: note = hTSH-A R primer
  source 1..21: mol_type = other DNA
gatctggacgaagagcatcag

SEQ ID NO: 8 - DNA, 21 bp, synthetic construct
  misc_feature 1..21: note = CreER F primer
  source 1..21: mol_type = other DNA
aaagtcgctctgagttgttat

SEQ ID NO: 9 - DNA, 19 bp, synthetic construct
  misc_feature 1..19: note = CreER R primer
  source 1..19: mol_type = other DNA
cctgatcctggcaatttcg

(19) United States
(12) Patent Application Publication
(10) Pub. No.: US 2024/0349706 A1
(43) Pub. Date: Oct. 24, 2024

(54) Novel Animal Model for Autoimmune Disease

(71) Applicant: INDUSTRY-ACADEMIC COOPERATION FOUNDATION, YONSEI UNIVERSITY, Seoul (KR)

(72) Inventors: Eun Jig Lee, Seoul (KR); Daham Kim, Seoul (KR)

(73) Assignee: INDUSTRY-ACADEMIC COOPERATION FOUNDATION, YONSEI UNIVERSITY, Seoul (KR)

(21) Appl. No.: 18/305,342

(22) Filed: Apr. 22, 2023

(51) Int. Cl. A01K 67/027; C07K 14/72; C12N 15/85

(52) U.S. Cl. CPC A01K 67/0278; C07K 14/723; C12N 15/8509; A01K 2217/072; A01K 2217/203; A01K 2227/105; A01K 2267/0325; C12N 2015/8563; C12N 2800/30

(57) ABSTRACT

The present invention relates to an autoimmune disease animal model and a method for preparing the same. The animal model of the present invention efficiently generates an autoantibody that causes autoimmune disease, by introducing an autoantigen-encoding gene through the Cre-LoxP system, which enables stable and continuous expression of the autoantigen at a desired time by treatment of Cre recombinase. Contrary to the conventional method that fails to effectively produce autoantibody due to leaky expression and immune tolerance, or requires repeated administration over several weeks, the present invention achieves sustainable expression of an effective amount of autoantigen by only one TAT-Cre recombinase treatment, thus may be utilized as an outstanding animal model reproducing various symptoms and molecular mechanisms of autoimmune diseases.

### CROSS-REFERENCE TO RELATED APPLICATIONS

This application claims the benefit of priority of Korean Patent Application no. 10-2022-0050016, filed Apr. 22, 2022, which is hereby incorporated herein by reference in its entirety.

### REFERENCE TO AN ELECTRONIC SEQUENCE LISTING

The instant application contains a Sequence Listing which has been submitted electronically and is hereby incorporated by reference in its entirety. The Sequence Listing was created on Apr. 10, 2023, is named “POPB222125US_seq.listings_ST26.xml” and is 28,187 bytes in size.

### TECHNICAL FIELD

The present invention relates to an animal model for autoimmune disease, specifically autoimmune thyroid disease, that continuously expresses an autoantigen at a desired time through the Cre-LoxP system.

### BACKGROUND OF THE INVENTION

Autoimmune disease refers to a disorder in which the immune system attacks self organs causing spontaneous reactions, resulting in tissue damage. Autoimmune diseases are characterized by a pathological response to autoantigens, and include systemic autoimmune diseases such as systemic lupus erythematosus, rheumatoid arthritis and multiple sclerosis, and organ-specific diseases such as myasthenia gravis, insulin-dependent diabetes, and Graves' disease.

Although an animal model capable of efficient reproduction of symptoms and mechanisms for autoimmune disease and responding to treatments developed for humans is essential for study of the pathogenesis and development of the therapeutic agent, an effective animal model has not been developed yet.

Meanwhile, Graves' disease is a type of autoimmune disease where the patient's own immune cells produce antibodies binding to thyroid cells. These antibodies stimulate the thyroid gland to produce excess thyroid hormone. The antibody also reacts to the tissue behind the eye, causing ocular symptoms such as exophthalmos and thyroid-associated ophthalmopathy. Previous studies reported the preparation of a Graves' disease model by introducing TSHR plasmid or adenovirus as an autoantigen to mice to cause an immune response, but it had limitations for the platform of new therapeutic agent development since it requires a lot of effort of repeated administrations for weeks, and the results were quite uneven. Animal models of other autoimmune diseases, such as myasthenia gravis, also require long-term repeated antigen administration, thus an effective model for autoimmune disease has not been developed yet despite the demands of the art.

Throughout this application, various publications and patents are referred and citations are provided in parenthesis. The disclosure of these publications and patents in their entities are hereby incorporated by references into this application in order to fully describe this invention and the state of the art to which this invention pertains.

### DISCLOSURE OF THE INVENTION

### Technical Problem

The present inventors have made intensive studies to develop a stable animal model that continuously self-expresses autoantigens, thus efficiently reflects various diseases caused by excessive autoantibodies or unwanted immune responses. As results, the present inventors have discovered that introduction of autoantigen gene into the animal through Cre-LoxP system enables the expression of the autoantigen at a desired time point only by treating Cre recombinase, leading to production of autoantibodies in an effective amount enough for the reproduction of the symptoms of autoimmune disease while avoiding immune tolerance and leaky expression.

Accordingly, it is an objective of this invention to provide a gene delivery system for preparing an animal in which autoimmune diseases are induced.

It is another objective of this invention to provide a method for preparing an animal in which autoimmune diseases are induced, and an autoimmune disease animal model prepared using the same.

Other objectives and advantages of the present invention will become apparent from the following detailed description together with the appended claims and drawings.

### Technical Solution

In one aspect of the present invention, there is provided a gene delivery system for preparing an animal in which an autoimmune disease is induced, comprising in a 5′ to 3′ direction, an expression regulatory sequence, an expression blocking nucleotide with loxp (locus of X-over P1) nucleotide sequence flanking on both sides, and a nucleotide encoding an autoantigen protein.

The present inventors have made intensive studies to develop a stable animal model that continuously self-expresses autoantigens, thus efficiently reflects various diseases caused by excessive autoantibodies or unwanted immune responses. As results, the present inventors have discovered that introduction of autoantigen gene into the animal through Cre-LoxP system enables the expression of the autoantigen at a desired time point only by treating Cre recombinase, leading to production of autoantibodies in an effective amount enough for the reproduction of the symptoms of autoimmune disease while avoiding immune tolerance and leaky expression.

The term “animal” as used herein refers to animals other than humans, and more concretely mammals other than humans.

The term “mammal” as used herein refers to all animals other than humans that belong to the mammalian family, and includes, for example, mice, rats, guinea pigs, dogs, cats, horses, cows, pigs, monkeys, chimpanzees, baboons, or rhesus monkeys, but is not limited thereto. Concretely, the mammal of the present invention is a rodent, and more concretely a mouse.

The term “animal model” as used herein refers to a non-human animal that has been artificially modified, directly or indirectly, to have the characteristics, phenotypes, or symptoms of autoimmune diseases, and more concretely refers to an animal that reproduces the overall biological processes driven by the production of autoantibodies through the introduction of a gene encoding an autoantigen protein via Cre-LoxP system, of which the expression is triggered upon treatment with Cre recombinase.

The term “nucleotide” as used herein has comprehensive meaning including deoxyribonucleotides (gDNA and cDNA) or ribonucleotide molecules existing in single-stranded or double-stranded form, and also includes analogs in which sugar or base sites are modified, unless otherwise specified (Scheit, Nucleotide Analogs, John Wiley, New York (1980); Uhlman and Peyman, Chemical Reviews, 90:543-584 (1990)). According to the present invention, the loxp nucleotide is the nucleotide sequence of the Lox p (locus of X-over P1) site of bacteriophage P1, a 34 bp long nucleotide in which 8 asymmetric sequences exist between 13 inverted complementary sequences at both ends. Cre recombinase recognizes and binds to the loxp nucleotide. More concretely, the loxp nucleotide of the present invention consists of the nucleotide of SEQ ID NO:1.

It would be obvious to the skilled artisan that the nucleotide sequences used in this invention are not limited to those listed in the appended Sequence Listings. Variations in nucleotides may not result in changes in the protein product. Such nucleic acids include nucleic acid molecules comprising functionally equivalent codons, or codons encoding the same amino acid (codon degeneracy), or codons encoding biologically equivalent amino acids.

Considering the biologically equivalent variations described hereinabove, the nucleic acid molecule of this invention may encompass sequences exhibiting substantial identity with the sequences listed in the appended Sequence Listings. Sequences exhibiting substantial identity show at least 60%, concretely at least 70%, more concretely at least 80%, and most concretely at least 90% similarity to the nucleic acid molecule of this invention, as measured using one of the sequence comparison algorithms well-known in the art when the sequence of the present invention and any other sequence are aligned so as to correspond as much as possible.

The term “expression blocking nucleotide” as used herein refers to a nucleic acid molecule located upstream of a target gene (i.e. nucleotide sequence encoding autoantigen protein) whose expression is to be controlled, that normally blocks the expression of the target gene by separating the expression regulatory sequence from the target gene. The expression blocking nucleotide is inactivated when Cre recombinase is treated, by loxp nucleotide sequences flanked on both sides thereof, thereby acting as a switch to initiate expression of the target gene. Expression blocking nucleotide includes, for example, DsRed2, green fluorescent protein (GFP), kanamycin resistance gene (kanaR), neomycin resistance gene (neoR), and combinations thereof, but are not limited thereto, and may be any nucleic acid sequence that is of appropriate length to prevent an expression regulatory sequence (e.g. a promoter) and a target gene from being operatively linked.

The term “gene carrier” or “gene delivery system” as used herein refers to any means of delivering a gene into a cell and expressing it in the cell. The gene delivery has the same meaning as intracellular transduction of genes. At the tissue level, the term gene delivery has the same meaning as the spread of a gene. Accordingly, the gene delivery system of the present invention can be described as a gene penetration system or a gene spread system

The nucleotide sequence of the present invention can be applied to all gene delivery systems used in conventional animal transformation, including, but not limited thereto, plasmids, adenovirus (Lockett L J, et al., Clin. Cancer Res. 3: 2075-2080 (1997)), adeno-associated virus (AAVs) (Lashford L S., et al., Gene Therapy Technologies, Applications and Regulations Ed. A. Meager, 1999), retrovirus (Gunzburg W H, et al., Retroviral vectors. Gene Therapy Technologies, Applications and Regulations Ed. A. Meager, 1999), lentiviruses (Wang G. et al., J. Clin. Invest. 104(11):R55-62(1999)), herpes simplex virus (Chamber R., et al., Proc. Natl. Act. Sci USA 92:1411-1415 (1995)), vaccinia virus (Puhlmann M. et al., Human Gene Therapy 10:649-657 (1999)), liposome (Methos in Molecular Biology, Vol 199, S. C. Basu and M. Basu (Eds.), Human Press 2002) or niosome. More concretely, the gene delivery system of this invention may be a plasmid.

Even more concretely, the gene delivery system used in the present invention may be a Rosa26 plasmid, and most concretely it is a Rosa26 vector having a genetic map shown in FIG. 1.

The term “express” as used herein refers to being artificially replicated as an extrachromosomal factor or by chromosomal integration in a target cell via a gene delivery system to cause the target cell to express an exogenous gene or to increase the natural expression level of an endogenous gene. Accordingly, the term “express” may be used interchangeably with “transformation”, “transfection”, or “transduction”.

To prepare the gene delivery system, the nucleotide sequence of this invention may be present in a suitable expression construct where the target gene is operatively linked to a promoter. The term “operably linked” as used herein refers to a functional linkage between a nucleic acid expression regulatory sequence (e.g., an array of promoter, signal sequence, or transcription factor binding positions) and target nucleic acid sequence, whereby the regulatory sequence controls the transcription and/or translation of said target nucleic acid sequence. The promoter linked to the target gene of the present invention is capable of operating in an animal cell, more concretely a mammalian cell, to regulate the transcription of the target gene, and includes the promoters derived from a mammalian virus or a genome of a mammalian cell, including, but not limited thereto, CMV (mammalian cytomegalovirus) promoter, adnovirus late promoter, vaccinia virus 7.5K promoter, SV40 promoter, tk promoter of HSV, RSV promoter, EF1 alpha promoter, metallothionein promoter, beta-actin promoter, promoter of human IL-2 gene, promoter of human IFN gene, promoter of human IL-4 gene, promoter of human lymphotoxin gene, and promoter of human GM-CSF gene.

According to a concrete embodiment of the present invention, the expression regulatory sequence used in this invention is a CAG promoter.

The term “autoimmune disease” as used herein refers to any pathological condition in which tissues or organs in the body are damaged or lose their functions due to excessive or unwanted humoral or cellular immune responses.

According to a concrete embodiment, the autoimmune disease to be reproduced in the animal of this invention includes rheumatoid arthritis, reactive arthritis, type 1 diabetes, type 2 diabetes, systemic lupus erythematosus, multiple sclerosis, idiopathic fibrosing alveolitis, myasthenia gravis, polymyositis, dermatomyositis, localized dermatosclerosis, systemic scleroderma, colitis, inflammatory bowel disease, Sjogren's syndrome, Raynaud's phenomenon, Bechet's disease, Kawasaki's disease, primary biliary sclerosis, primary sclerosing cholangitis, ulcerative olitis, Crohn's disease, and autoimmune immune thyroid disease, but are not limited thereto.

According to a concrete embodiment of the present invention, the autoimmune disease is an autoimmune thyroid disease.

The term “autoimmune thyroid disease” as used herein refers to a disease in which thyroid hormones are over-secreted or deficient due to an immune response mediated by autoantibodies against the thyroid gland. When a thyroid stimulating antibody (TSAb) for thyroid hormone receptor is generated, thyroid hormone is overproduced resulting in progression to hyperthyroidism, whereas when a TSH-stimulation blocking antibody (TSBAb) against thyroid hormone receptors or a thyroid peroxidase antibody (TPOAb) that destroys thyroid cells are produced, hormone secretion decreases and hypothyroidism develops.

More concretely, the autoimmune thyroid disease is selected from the group consisting of Graves's disease, Hashimoto's thyroiditis, atrophic thyroiditis, painless thyroiditis, and postpartum thyroiditis.

Most concretely, the autoimmune thyroid disease is Graves' disease. Graves' disease is a pathogenic condition where the patient's own immune cells produce antibodies binding to thyroid cells, resulting in hypersecretion of thyroid hormones. It is characterized by three symptoms: eyeball protrusion, thyroid enlargement, and heart palpitations.

According to a concrete embodiment of the present invention, the autoantigen protein used in this invention is thyroid stimulating hormone receptor (TSHR) or a functional portion thereof. The term “functional portion of TSHR” refers to an equivalent fragment of full-length protein form where certain amino acid residues are deleted or a partial domain of the full-length protein, which maintains the original biological activity and function of the full-length protein. More concretely, it refers to some fragment or domain that retains the immunogenicity of the full-length of TSHR protein. Concretely, the “functional portion of TSHR” may be a TSHR alpha subunit.

In another aspect of this invention, there is provided a method for preparing an animal in which autoimmune diseases are induced comprising introducing the aforementioned gene delivery system of this invention into an animal.

The terms “introducing the gene delivery system into an animal” refers to enabling the target gene being artificially replicated as an extrachromosomal factor or by chromosomal integration through transporting a gene delivery system into which a target nucleic acid molecule is inserted into the cells of an animal, in order to artificially express a target nucleic acid molecule in the animal.

The introduction of the gene delivery system may be performed using various animal transduction methods well-known in the art. For example, when the gene delivery system of the present invention is prepared based on a viral vector, the contacting step is performed according to a virus infection method known in the art.

When the gene delivery system in the present invention is a naked recombinant DNA molecule or plasmid, the gene delivery system can be introduced into the cells by way of the microinjection method (Capecchi, M. R., Cell, 22:479 (1980); and Harland et al., J. Cell Biol. 101:1094-1099 (1985)), calcium phosphate precipitation (Graham, F. L. et al., Virology, 52:456 (1973); and Chen and Okayama, Mol. Cell. Biol. 7:2745-2752 (1987)), biography perforation (Neumann, E. et al., EMBO J., 1:841 (1982); and Tur-Kaspa et al., Mol. Cell Biol., 6:716-718 (1986)), liposome-mediated transformation Infection methods (Wong, T. K. et al., Gene, 10:87 (1980); Nicolau. etene, Biochim. Biophys. Acta, 721:185-190 (1982); and Nicolau. et al., Methods Enzymol., 149:157-176 (1987)), DEAE-dextran treatment (Gopal, Mol. Cell Biol., 5:1188-1190 (1985)), and gene bombardment (Yang et al., Proc. Natl. Acad. Sci., 87:9568-9572 (1990)).

According to a concrete embodiment, the method of the present invention may be performed by injecting embryonic stem cells into which the gene delivery system has been introduced into the animal. An animal model expressing autoantigen proteins at the desired time point through the treatment of Cre recombinase may be obtained by injecting embryonic stem cells introduced with the gene delivery system of this invention into the fertilized eggs of the animal. For example, TSHR knock-in animal may be obtained by cleaving with restriction enzymes to linearize the circular plasmid, separating the target site including the target gene by electrophoresis and inserting into embryonic stem cells, and then mating the chimera mice prepared by transplanting the embryonic stem cell into fertilized eggs.

According to a concrete embodiment, the method of this invention further comprises repeating backcrosses between an animal into which the gene delivery system has been introduced and the recurrent parent 2 to 6 times. More concretely, backcrossing may be repeated 3 to 6 times, more concretely 4 to 6 times, and most concretely 5 or 6 times.

According to a concrete embodiment of the present invention, the method of this invention further comprises treating TAT-Cre recombinase or introducing a nucleotide encoding Cre recombinase into an animal into which the gene delivery system has been introduced.

In conventional animal models where autoantigen genes were simply overexpressed, autoantibodies were not produced due to immune tolerance. The Tamoxifen-induced Cre-loxp system prepared by crossing animals with a Cre recombinase gene and animals with loxP sequence inserted, was expected to induce the expression of the target gene through activation of Tamoxifen-inducible Cre at a desired time by tamoxifen injection, but did not produce an effective amount of autoantibody due to leaky expression and immune tolerance. In contrast, the animal model of this invention enables selecting the time of autoantigen expression as needed by a simple process of treating TAT-Cre recombinase or introducing Cre recombinase-encoding genes by a known gene transfer method (e.g., electroporation using a plasmid or viral vector, etc.) to an animal where target gene and loxP sequence are knocked in. The present invention provides efficient expression of an optimal amount of autoantigens suitable for autoantibody production to the extent that autoimmune diseases can be reproduced without leaky expression.

According to a concrete embodiment of the present invention, the TAT-Cre recombinase is administered at 50-1500 U. More concretely, it is administered at 100-1250 U, more concretely at 200-1000 U, and most concretely at 250-750 U.

In another aspect of the present invention, there is provided an animal in which an autoimmune disease is induced, prepared by the aforementioned method of the present invention.

In another aspect of the present invention, there is provided an animal induced with autoimmune disease, comprising an expression-blocking nucleotide with a loxp (locus of X-over P1) nucleotide sequence flanked on both sides, and a nucleotide encoding an autoantigen protein.

Since the gene delivery vehicle, the animal model into which it was introduced, and the autoimmune diseases to be reproduced in this invention have already been described above in detail, descriptions thereof are omitted to avoid excessive redundancy.

### Effects of the Invention

The features and advantages of the present invention are summarized as follows:
(a) The present invention provides an autoimmune disease animal model and a method for preparing the same.
(b) The animal model of the present invention efficiently generates an autoantibody that causes autoimmune disease, by introducing an autoantigen-encoding gene through the Cre-LoxP system, which enables stable and continuous expression of the autoantigen at a desired time by treatment of Cre recombinase.
(c) Contrary to the conventional method that fails to effectively produce autoantibody due to leaky expression and immune tolerance, or requires repeated administration over several weeks, the present invention achieves sustainable expression of an effective amount of autoantigen by only one TAT-Cre recombinase treatment, thus may be utilized as an outstanding animal model reproducing various symptoms and molecular mechanisms of autoimmune diseases.

### BRIEF DESCRIPTION OF THE DRAWINGS

FIG. 1 represents a diagram showing the plasmid map of Rosa26_hTSHRa, where hTSHRa is inserted into the Cre-LoxP system.

FIG. 2A represents a diagram showing the insertion of necessary parts of the Rosa26_hTSHRa plasmid into the ROSA26 gene region of embryonic stem cells via homologous recombination. FIG. 2B represents the results of RT-PCR confirming at RNA level that, while hTSHRa is not normally expressed in the cell lines produced by inserting necessary parts of the Rosa26_hTSHRa plasmid into mouse embryonic stem cells, it is expressed only when Cre recombinant enzyme is introduced by the transduction of nls-Cre plasmid.

FIG. 3 represents a schematic diagram outlining the manufacturing process for the autoimmune disease animal model of this invention. As the first method, B6;129S-Gt (ROSA)26Sor^{tm1(cre/ERT2/hTSHRa)} were obtained by crossing with B6;129S-Gt (ROSA)26Sor^{tm1(hTSHRa)} and then tamoxifen was administered. As the second method, TAT-Cre was administered to B6;129S-Gt (ROSA)26Sor^{tm1(hTSHRa)} mice. As the third method, B6;129S-Gt (ROSA)26Sor^{tm1(hTSHRa)} mice were backcrossed with BALB/c mice to create BALBc;129S-Gt (ROSA)26Sor^{tm1(hTSHRa)} mice, and TAT-Cre was administered to BALBc;129S-Gt (ROSA)26Sor^{tm1(hTSHRa)} mice.

FIG. 4 represents the expression of TSHR Ab (antithyroid receptor antibody) (FIG. 4a) and increase of thyroid hormone (T4) (FIG. 4b) at 12 weeks after the administration of various concentrations of tamoxifen to B6;129S-Gt (ROSA)26Sor^{tm1(hTSHRa)} mice.

FIG. 5 represents the expression of TSHR Ab (FIG. 5a) and increase of T4 (FIG. 5b) at 12 weeks after the administration of various concentrations of TAT-Cre to B6;129S-Gt (ROSA)26Sor^{tm1(hTSHRa)} mice.

FIG. 6 represents the expression of TSHR Ab (FIG. 6a) and increase of T4 (FIG. 6b) at 12 weeks after the administration of various concentrations of TAT-Cre to B6;129S-Gt (ROSA)26Sor^{tm1(hTSHRa)} mice and BALBc;129S-Gt (ROSA)26Sor^{tm1(hTSHRa)} mice.

FIG. 7 represents the expression of TSHR Ab (TSHR Ab) and increase of T4 at 4 weeks (FIG. 7a), 8 weeks (FIG. 7b), and 12 weeks (FIG. 7c) after the administration of 500 U TAT-Cre to 8 BALBc;129S-Gt (ROSA)26Sor^{tm1(hTSHRa)} mice (homozygous) and 100 U TAT-Cre to 5 BALBc;129S-Gt (ROSA)26Sor^{tm1(hTSHRa)} mice.

FIG. 8 represents the expression of TSHR Ab (FIG. 8a) and increase of T4 (FIG. 8b) at 4, 8, 12, and 20 weeks after the administration of 500 U TAT-Cre to 2 BALBc;129S-Gt (ROSA)26Sor^{tm1(hTSHRa)} mice (homozygous).

FIG. 9 represents the results of a histological study of the thyroid gland at 13 weeks after administering 500 U TAT-Cre to 8 BALBc;129S-Gt(ROSA)26Sor^{tm1(hTSHRa)} mice (homozygous). FIG. 9a shows the representative images of the thyroids. FIG. 9b shows the representative images of the thyroid glands stained with hematoxylin and eosin (H&E) (magnification, 200×). FIG. 9c shows the representative images of the thyroid glands stained with CD3 antibody (magnification, 100×). The bar at the lower right of the images represents 50 μm.

FIGS. 10A, 10B, 10C and 10D represent the results of a histological study of the orbital tissue at 13 weeks after administering 500 U TAT-Cre to 8 BALBc;129S-Gt(ROSA)26Sor^{tm1(hTSHRa)} mice (homozygous). FIG. 10A shows the representative images of the orbital tissue stained with hematoxylin and eosin (H&E) (magnification, 100×). FIG. 10B shows the representative images of the orbital muscle tissue F4/80+ staining (magnification, 400×). FIG. 10C shows the representative images of the orbital muscle tissue Masson's trichrome staining (magnification, 400×). FIG. 10A shows the representative images of the orbital muscle tissue Alcian blue staining (magnification, 400×).

The bar at the lower right of the images represents 50 μm.

Hereinafter, the present invention will be described in further detail by examples. It would be obvious to those skilled in the art that these examples are intended to be more concretely illustrative and the scope of the present invention as set forth in the appended claims is not limited to or by the examples

### Examples

### 1. Construction of Rosa26_hTSHRa Vector and Insertion of Mouse Embryonic Stem Cells

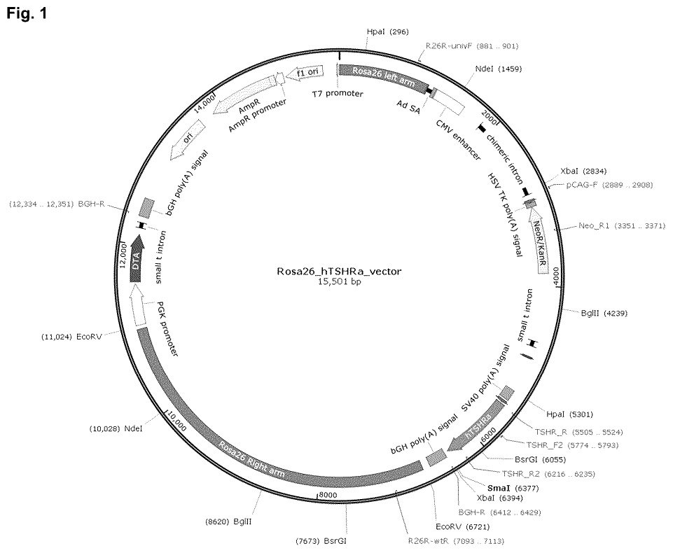

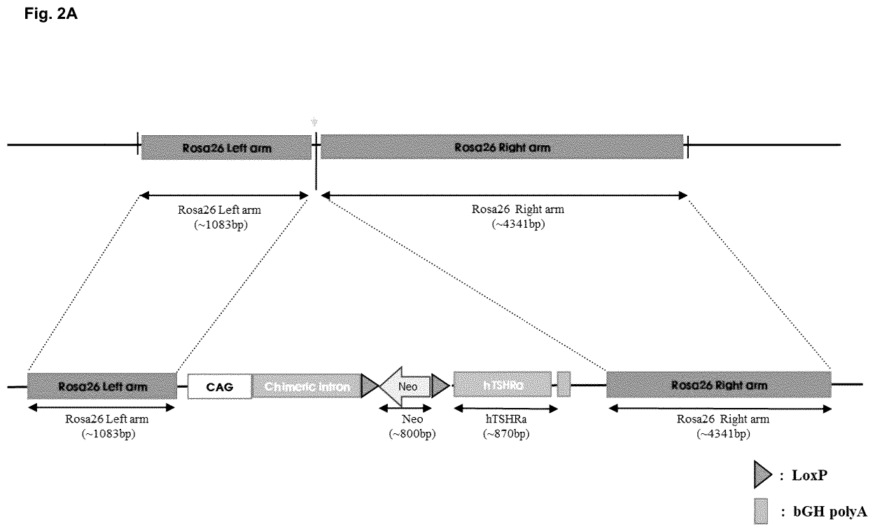

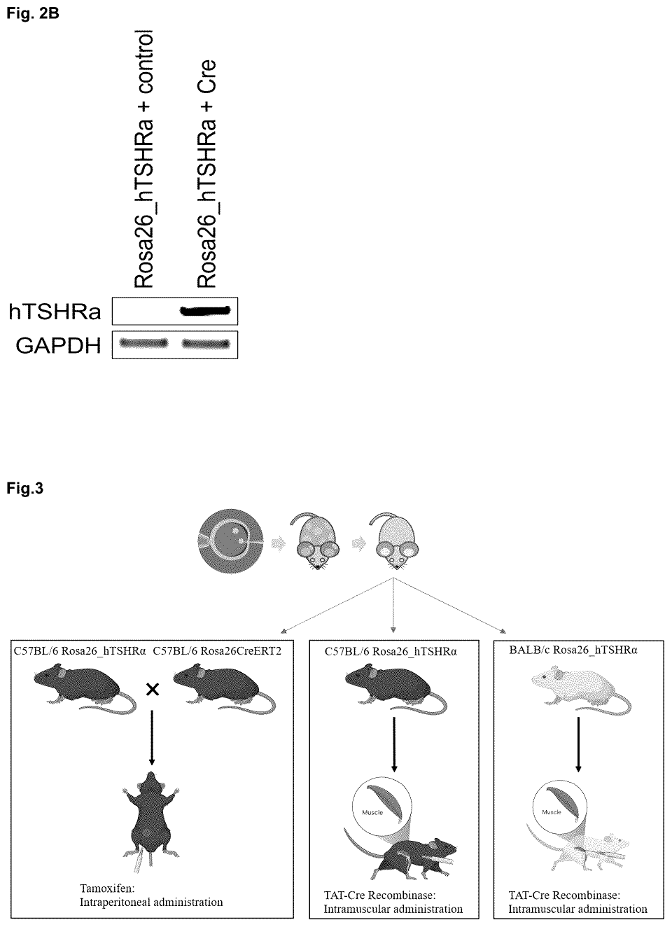

The TSHRα subunit (289) cDNA from human normal thyroid tissue was used for cloning. The Kozak sequence GCCACC, human TSHRα subunit (289), and stop codon TAA sequences were inserted into the Rosa26 vector (the Cre-LoxP system which is not normally expressed, but is expressed when Cre is treated) using AscI and XmaI (New England Biolabs, Ipswich, MA, USA) restriction site cloning to construct the Rosa26_hTSHRa vector (FIG. 1). Then, the Rosa26_hTSHRa vector was linearized by digestion with restriction enzymes, and the necessary parts were separated by electrophoresis, inserted into 129S1/SvImJ mouse embryonic stem cells by homologous recombination, selected with neomycin, and then sequenced to confirm the sequence (FIG. 2A). Before the production of the knock-in mouse, it was confirmed in RNA level that hTSHRa is expressed only when Cre recombinant enzyme is introduced by the transduction of nls-Cre plasmid, although hTSHRa is not normally expressed at the recombinant embryonic stem cell line stage (FIG. 2B).

2. Production of human TSHRα Subunit Knock-In Mice

### (1) B6;129S-Gt (ROSA)26Sor^{tm1(hTSHRa)}

Confirmed recombinant embryonic stem cells were microinjected into fertilized mouse eggs, and the surviving fertilized eggs were selected and transplanted into the fallopian tube of a surrogate mouse, by which delivered chimeric mice were obtained. Then, the backcrossing method was applied to C57BL/6 mice (recurrent parents). The F1 progeny obtained had 50% of the genetic material of each parent. After the mouse grew for about 2 weeks, the tail was cut and DNA was extracted to confirm whether the hTSHRα subunit was inserted. F1 individuals were crossed with the recurrent parents to obtain F2 individuals. The F2 individuals were then crossed with the recurrent parents. The proportion of donor parents decrease in half in each backcrossed generation. Since more than 96% of the mice are identical to the recurrent parent after the F5 generation, mice of at least or after the F5 generation were used for the experiment.

### (2) B6;129S-Gt (ROSA)26Sor^{tm1(cre/ERT2/hTSHRa)}

B6;129S-Gt (ROSA)26Sor^{tm1(hTSHRa)} of the F5 or later generation having hTSHRα subunit gene was crossed with the B6;129S-Gt (ROSA)26Sor^{tm1(cre/ERT2)} mice, to which the tamoxifen-induced Cre-mediated recombination system had been introduced via the endogenous mouse Gt(ROSA)26Sor promoter, to create B6;129S-Gt (ROSA)26Sor^{tm1(cre/ERT2/hTSHRa)} mice.

### (3) BALBc;129S-Gt (ROSA)26Sor^{tm1(hTSHRa)}

B6;129S-Gt (ROSA)26Sor^{tm1(hTSHRa)} mice were backcrossed to BALB/c mice (recurrent parents). Obtained F1 progeny had 50% of the genetic material of each parent, and after the mouse grew for about 2 weeks, the tail was cut and DNA was extracted to confirm whether the hTSHRα subunit was inserted. F1 individuals were crossed with the recurrent parents to obtain F2 individuals. The F2 individuals were then crossed with the recurrent parents. The proportion of donor parents decrease in half in each backcrossed generation. Since more than 96% of the mice are identical to the recurrent parent after the F5 generation, mice of at least or after the F5 generation were used for the experiment.

### 3. Genotyping Target Mouse Strains

B6;129S-Gt (ROSA)26Sor^{tm1(hTSHRa)} and BALBc;129S-Gt (ROSA)26Sor^{tm1(hTSHRa)}

TABLE 1

| Target | | |
|---|---|---|
| Gene | Primer | Sequence |
| Rosa 26 | Forward | 5′-AAA GTC GCT CTG AGT TGT TAT-3′ |
| | Reverse | 5′-GGA GCG GGA GAA ATG GAT ATG-3′ |
| hTSH-A | Forward | 5′-GCA ACG TGC TGG TTA TTG TG-3′ |
| | Reverse | 5′-GAT CTG GAC GAA GAG CAT CAG-3′ |

The B6;129S-Gt (ROSA)26Sor^{tm1(hTSHRa)} and BALBc;129S-Gt (ROSA)26Sor^{tm1(hTSHRa)} mice were grown for about two weeks, and the tails of the mice were cut for DNA extraction. PCR was performed using the above primers to obtain a 483 base pair band at the target locus and a 603 base pair band at the wild type locus. PCR was performed for 34 cycles at 94° C. for 30 seconds, 65° C. for 60 seconds and 72° C. for 60 seconds.

### B6;129S-Gt (ROSA)26Sor^{tm1(cre/ERT2/hTSHRa)}

TABLE 2

| Target | | |
|---|---|---|
| Gene | Primer | Sequence |
| Rosa 26 | Forward | 5′-AAA GTC GCT CTG AGT TGT TAT-3′ |
| | Reverse | 5′-GGA GCG GGA GAA ATG GAT ATG-3′ |
| CreER | Forward | 5′-AAA GTC GCT CTG AGT TGT TAT-3′ |
| | Reverse | 5′-CCT GAT CCT GGC AAT TTC G-3′ |
| hTSH-A | Forward | 5′-GCA ACG TGC TGG TTA TTG TG-3′ |
| | Reverse | 5′-GAT CTG GAC GAA GAG CAT CAG-3′ |

The B6;129S-Gt (ROSA)26Sor^{tm1(cre/ERT2/hTSHRa)} mice were grown for about two weeks, and the tails of the mice were cut for DNA extraction. PCR was performed using the above primers to obtain a 483 base pair band and 823 base pair band at the target locus and a 603 base pair band at the wild type locus. PCR was performed for 34 cycles at 94° C. for 30 seconds, 65° C. for 60 seconds and 72° C. for 60 seconds.

### 4. Measurement of Serum Antibodies and Thyroid Function

The expression of antithyroid receptor antibody (TSHR Ab) and thyroid hormone (T4) level were measured in the serum of each mouse. TSHR Ab was measured using the antithyroid receptor antibody from Fast ELISA kit (EUROIMMUN, Luebeck, Germany), and thyroid hormone (T4) was measured by ELISA (DRG, Springfield, NJ, USA). The tests were performed according to the manufacturer's instructions.

5. Induction of hTSHRα Subunit Expression in Transgenic Mice

Pathogen-free transgenic mice (B6;129S-Gt (ROSA)26Sor^{tm1(hTSHRa)} mice, BALBc;129S-Gt (ROSA)26Sor^{tm1(hTSHRa)} mice, and B6;129S-Gt (ROSA)26Sor^{tm1(cre/ERT2/hTSHRa)}) were bred under a 12-hour light and dark cycle while controlling temperature (24±2° C.) and humidity (55±15%). The mice were maintained under pathogen-free conditions in single ventilated cases to block environmental exposure. Before the start of the experiment, the mice were transferred to a clean environment from a SPF environment. Experiments were conducted using 6-8 weeks mice. All experiments were performed according to protocols approved by the Animal Research Ethics Committee of Yonsei University (Seoul, Korea).

### (1) Tamoxifen Administration

As a first method, tamoxifen was administered to B6;129S-Gt (ROSA)26Sor^{tm1(cre/ERT2/hTSHRa)} mice, which were obtained by crossing B6;129S-Gt (ROSA)26Sor^{tm1(hTSHRa)} and B6;129-Gt (ROSA)26Sor^{tm1(cre/ERT2)} mice (FIG. 3).

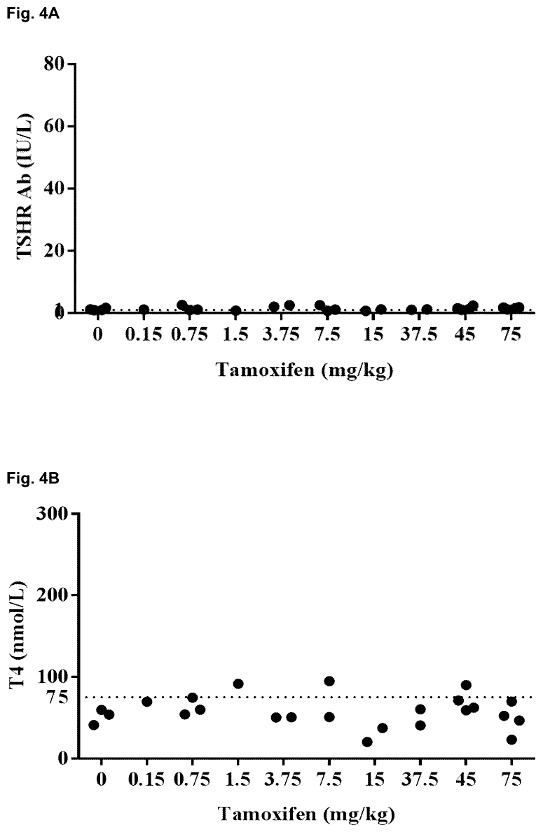

Tamoxifen (Sigma-Aldrich, St. Louis, MO, USA) dissolved in corn oil in a concentration of 10 mg/ml was stirred overnight at 37° C. Since tamoxifen is sensitive to light, it was stored at 4° C. in a light-protected container. To search an appropriate concentration range, the various dosages were tried at 0.15-75 mg tamoxifen/kg body weight. The injection site was sensitized with 70% ethanol, and then tamoxifen was intraperitoneally injected into B6;129S-Gt (ROSA)26Sor^{tm1(cre/ERT2/hTSHRa)} mice. The TSH R Ab expression and the level of thyroid hormone (T4) were measured in week 12, and no changes were observed compared to the control group (0 mg/kg) (FIG. 4A, FIG. 4B). It seems that antibodies are not produced well due to immune tolerance caused by leaky expression.

### (2) Administration of TAT-Cre Recombinase

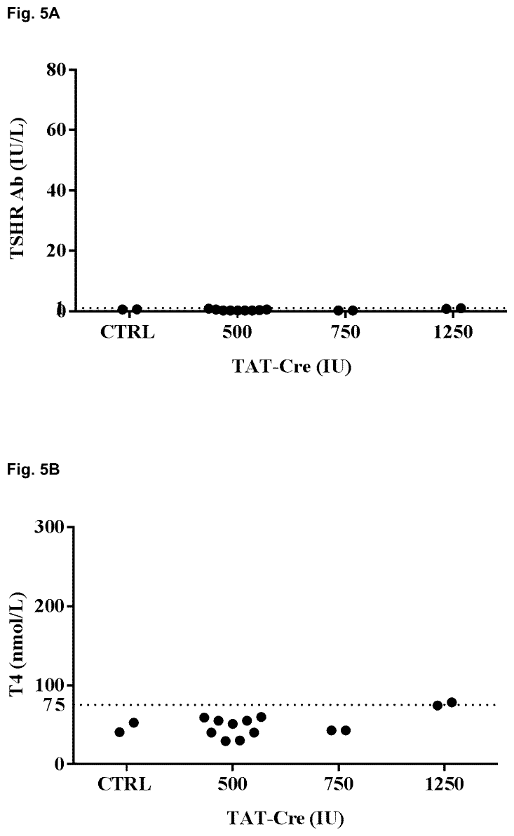

In order to determine whether the reason for insufficient antibody production is the immune tolerance due to leaky expression, TAT-Cre recombinase (Sigma-Aldrich, St. Louis, MO, USA) was injected intramuscularly at a concentration of 500-1250 U into the leg muscles of B6;129S-Gt (ROSA)26Sor^{tm1(cre/ERT2/hTSHRa)} mice. The injection site was sensitized with 70% ethanol before injection. The TSHR Ab expression and the level of thyroid hormone (T4) were measured in week 12, and no changes were observed compared to the control group (0 mg/kg) (FIG. 5A, FIG. 5A). These results indicate that the B6;129S-Gt (ROSA)26Sor^{tm1(cre/ERT2/hTSHRa)} mice did not produce antibodies well due to immune tolerance caused by leaky expression.

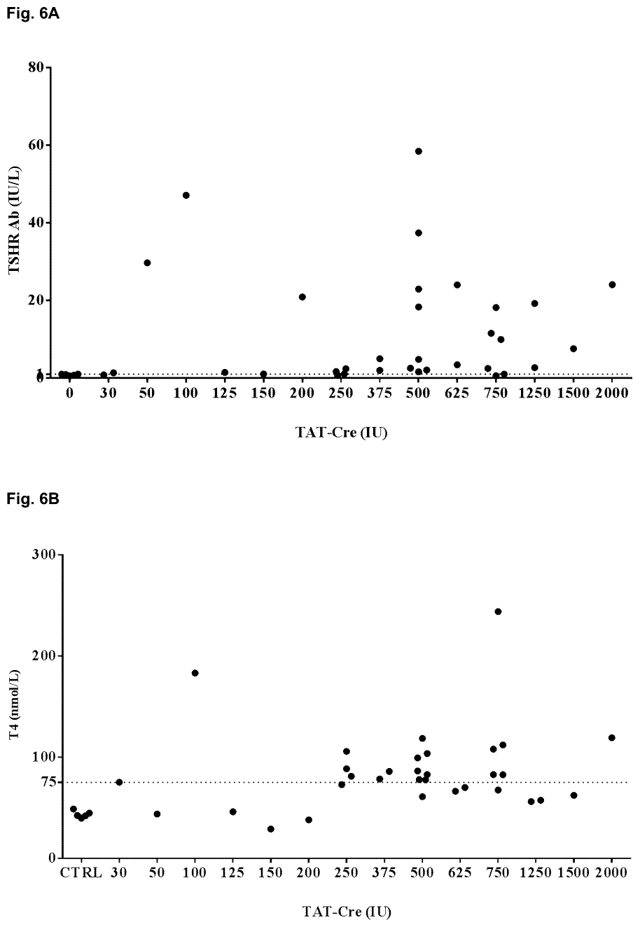

In order to completely avoid immune tolerance due to leaky expression, various concentrations of TAT-Cre recombinase (Sigma-Aldrich, St. Louis, MO, USA) from 30 to 2000 U were intramuscularly injected into the leg muscles of B6;129S-Gt (ROSA)26Sor^{tm1(hTSHRa)} mice and BALBc;129S-Gt (ROSA)26Sor^{tm1(hTSHRa)} mice. The injection site was sensitized with 70% ethanol before injection. To search an appropriate concentration range, both heterozygous and homozygous of B6;129S-Gt (ROSA)26Sor^{tm1(hTSHRa)} mice and BALBc;129S-Gt (ROSA)26Sor^{tm1(hTSHRa)} mice were used. The TSHR Ab expression and the level of thyroid hormone (T4) were measured in week 12. As results, TSHR Ab production and increase of thyroid hormone (T4) were observed at various concentrations of TAT-Cre (FIGS. 6a and 6b), from which it is confirmed that this method can be used to create an animal model for Graves' disease, a representative organ-specific autoimmune disorder.

At a TAT-Cre dose less than 30 U, an antigen-antibody reaction was not sufficiently induced, and at a dose of 100 U or more, an effective amount of antibody was produced and the level of thyroid hormone increased. Within the 250-750U range of TAT-Cre, the antibody was produced more efficiently and thyroid hormone significantly increased.

However, when the dose of TAT-Cre exceeded 1500 U, antibody neutralization was induced by contact with many antigens.

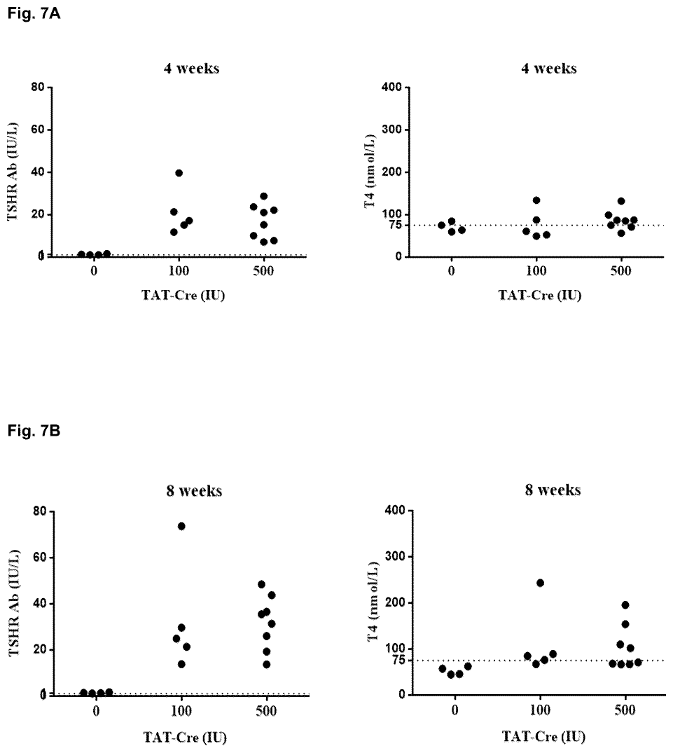

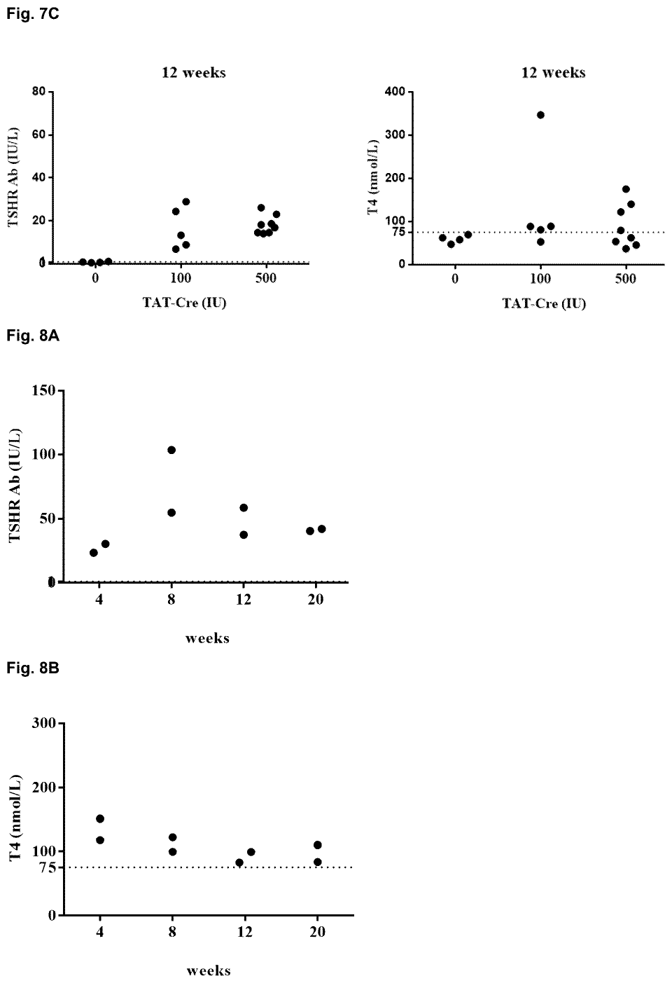

Subsequently, as an independent experiment, TAT-Cre recombinase (Sigma Aldrich, St. Louis, MO, USA) was intramuscularly injected at a concentration of 500 U into the leg muscles of 8 B6;129S-Gt (ROSA)26Sor^{tm1(hTSHRa)} mice. Other 5 mice were intramuscularly injected with a concentration of 100 U. The injection site was sensitized with 70% ethanol before injection. TSHR Ab expression and thyroid hormone (T4) were measured in week 4. As results, all mice administered with TAT-Cre significantly produced TSHR Ab, and thyroid hormone began to increase compared to the control group (OU). In week 8 and 12, TSHR Ab was produced at a very high level in all mice administered with TAT-Cre, and thyroid hormone (T4) highly increased in more than half of the mice administered with 500 U compared to the control group (OU) (FIGS. 7a, 7b and 7c). These results were even more remarkable than those in the group administered with 100 U.

As an independent experiment, TAT-Cre recombinase (Sigma Aldrich, St. Louis, MO, USA) was intramuscularly injected at a concentration of 500 U into the leg muscles of 2 BALBc;129S-Gt (ROSA)26Sor^{tm1(hTSHRa)} mice (homozygous). The injection site was sensitized with 70% ethanol before injection. When the expression of TSHR Ab and the level of thyroid hormone (T4) were measured in week 4, 8 and 12, the production of TSHR Ab and increase of thyroid hormone (T4) continued for more than 20 weeks (FIGS. 8a and 8b).

In conclusion, while TAT-Cre may be administered at various doses, an effective amount of antibody is generated at 50 U or more. In case it is administered at 1500 U or more, the problems including antibody neutralization due to excessive antigen production, and frequent administrations may arise. Therefore, administration of 50-1500 U, more concretely 100-1250 U, even more concretely 200-100 U, and most concretely 250-750 U was found to be appropriate.

### 6. Histological Study of the Thyroid Gland in a Graves' Disease Animal Model

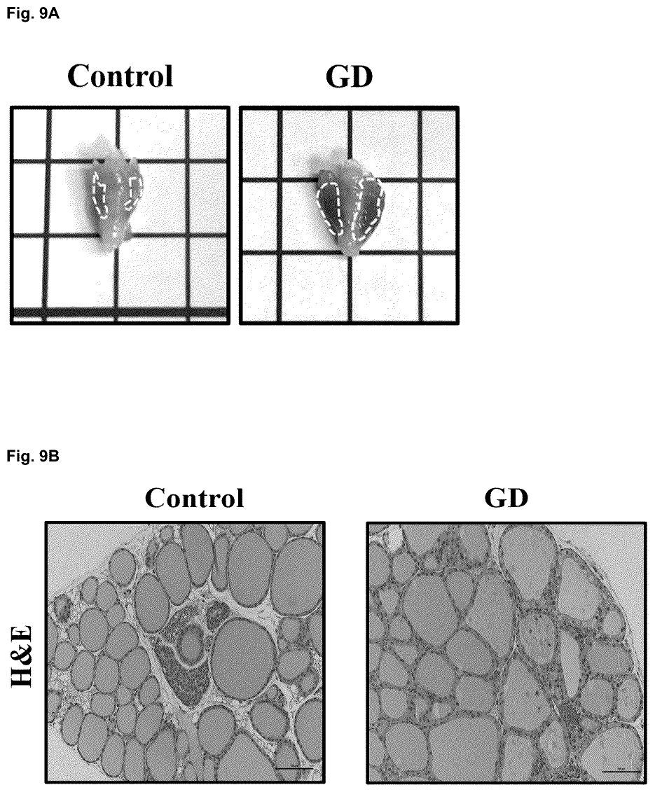

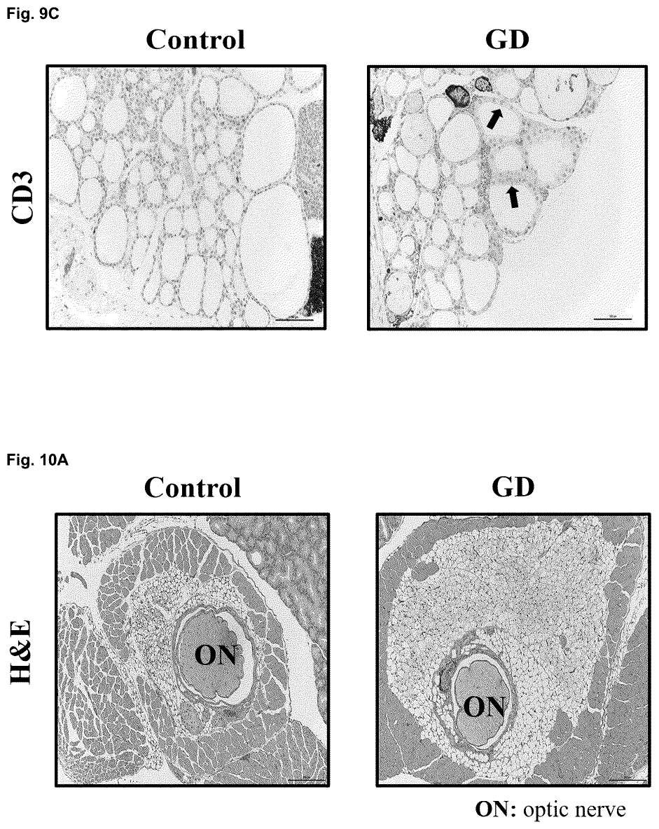

Contrary to the control group, the thyroid gland of the TAT-Cre treated mice (GD group) showed diffuse enlargement and hypertrophy (FIG. 9A). Examination of the thyroid glands via H&E staining revealed differences between the two groups. The normal thyroid tissue appears as closely packed follicles consisting of a single layer of thyroid follicular cells surrounding the lumen. The thyroid glands of TAT-Cre treated mice exhibited more columnar and vacuolated morphology compared to control mice, which is consistent with the presence of hyperthyroidism (FIG. 9A). Furthermore, immunohistological staining for CD3 revealed extensive T cell infiltration into interfollicular connective tissue in TAT-Cre treated mice (FIG. 9C).

### 7. Histological Study of the Orbit Tissue in a Graves' Disease Animal Model

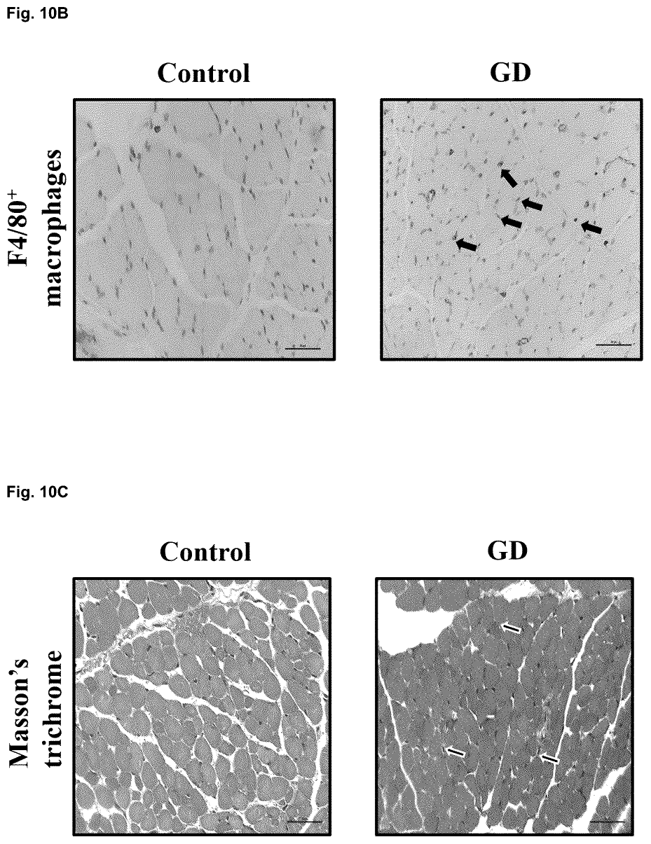

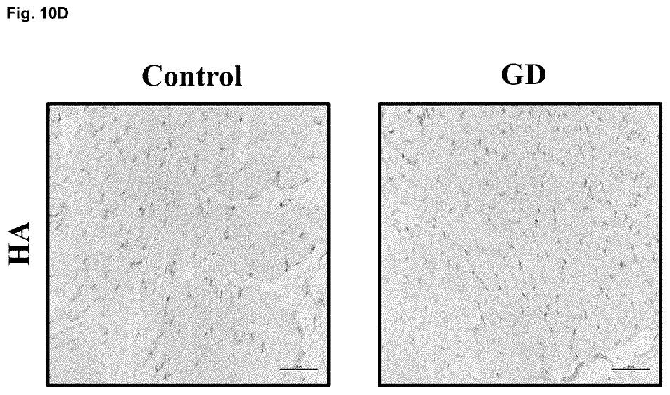

In the studies on the retrobulbar histopathology, the optic nerve was marked in all exhibited H&E sections to assist in the histopathological analysis. As revealed by an H&E examination of the orbital tissues, the retrobulbar tissue in the control group had a normal appearance but the retrobulbar adipose tissue was increased in TAT-Cre treated mice (FIG. 10A). Additionally, the orbital tissue underwent IHC for certain histological analyses. On IHC test, F4/80+ macrophages were more visible in the orbital tissue of TAT-Cre treated mice than in the orbital tissue of the control group (FIG. 10B).

After Masson's trichrome staining, the analysis of the TAT-Cre treated mice orbital tissue revealed fibrosis in muscle tissue. No abnormalities were found on the examination of the orbital tissue of the control group (FIG. 10C). Alcian blue staining of the orbital tissue showed no difference between the two groups (FIG. 10D).

Having described specific embodiment of the present invention in detail above, it is to be understood that variants and modifications thereof falling within the spirit of the invention may become apparent to those skilled in this art, and the scope of this invention is to be determined by appended claims and their equivalents.

1. A gene delivery system for preparing an animal in which an autoimmune disease is induced, comprising in a 5′ to 3′ direction, an expression regulatory sequence, an expression blocking nucleotide with loxp (locus of X-over P1) nucleotide sequence flanking on both sides, and a nucleotide encoding an autoantigen protein.

2. The gene delivery system according to claim 1, wherein the expression regulatory sequence is a CAG promoter.

3. The gene delivery system according to claim 1, wherein the expression blocking nucleotide is selected from the group consisting of DsRed2, GFP (green fluorescent protein), kanaR (kanamycin resistance gene), neoR (neomycin resistance gene), and combinations thereof.

4. The gene delivery system according to claim 1, wherein the autoimmune disease is an autoimmune thyroid disease.

5. The gene delivery system according to claim 4, wherein the autoimmune thyroid disease is selected from the group consisting of Graves' disease, Hashimoto's thyroiditis, atrophic thyroiditis, painless thyroiditis, and postpartum thyroiditis.

6. The gene delivery system according to claim 1, wherein the autoantigen protein is a TSHR (thyroid stimulating hormone receptor) or a functional portion thereof.

7. The gene delivery system according to claim 1, wherein the gene delivery system is a Rosa26 plasmid having the genetic map shown in FIG. 1.

8. The gene delivery system according to claim 1, wherein the animal is a rodent.

9. A method for preparing an animal in which an autoimmune disease is induced comprising introducing the gene delivery system according to claim 1 into an animal.

10. The method according to claim 9, wherein the method is performed by injecting embryonic stem cells into which the gene delivery system has been introduced into the animal.

11. The method according to claim 9, wherein the method further comprises repeating backcrosses between an animal into which the gene delivery system has been introduced and the recurrent parent 2 to 6 times.

12. The method according to claim 9, wherein the method further comprises treating TAT-Cre recombinase or introducing a nucleotide encoding Cre recombinase to an animal into which the gene delivery system has been introduced.

13. The method according to claim 12, wherein the TAT-Cre recombinase is treated at 50-1500 U.

14. The method according to claim 9, wherein the animal is a rodent.

15. An animal in which an autoimmune disease is induced, prepared by the method according to claim 9.

16. An animal in which an autoimmune disease is induced, comprising an expression blocking nucleotide with loxp (locus of X-over P1) nucleotide sequence flanking on both sides, and a nucleotide encoding an autoantigen protein.
